(19) United States
(12) Patent Application Publication
(10) Pub. No.: US 2006/0155276 A1
(43) Pub. Date: Jul. 13, 2006

(54) Bone fixation assembly and related method

(76) Inventors: Stephen B. Walulik, Phillipsburg, NJ (US); Kirk J. Bailey, Blairstown, NJ (US); Tim Elghazaly, Piscataway, NJ (US); Takkwong R. Leung, Piscataway, NJ (US)

Correspondence Address: HARNESS, DICKEY & PIERCE, P.L.C., P.O. BOX 828, BLOOMFIELD HILLS, MI, 48303, US

(21) Appl. No.: 11/032,867

(22) Filed: Jan. 11, 2005

(51) Int. Cl. A61B 17/60

(52) U.S. Cl. 606/59

(57) ABSTRACT

A fixation assembly for securing a first bone portion in fixed relationship relative to a second bone portion. The fixation assembly includes a plurality of modular components articulatably coupled therebetween, at least one fastener interconnecting two of the plurality of components, and a clamp connector adapted for selective unilateral or multilateral bone fixation. The entire fixation assembly or portions thereof can be selectively radiolucent.

In various orthopedic surgical procedures, it is often necessary to secure two or more portions of bone in a relatively fixed relationship to each other. This need is often a result of a fracture which has occurred to the bone as a result of trauma or from the correction of a deformity. To ensure that the bone can properly regenerate and fuse the fractures of the bone, it is important that the various bone portions be fixed at the desired position during bone regeneration.

Various external fixators for the repair of bone are known. Known fixators, although effective in fixating bones, generally do not to permit full radiographic examination of the repair site without removing the apparatus, typically providing only a limited radiographic view of the fracture site. Additionally, such fixators are adapted for use only for a particular type of fixation configuration. Thus, it would be advantageous to provide an external fixator that provides full radiographic view of the repair area and/or can be used for different types of fixation configurations.

### SUMMARY

The present teachings provide a fixation assembly for securing a first bone portion in fixed relationship relative to a second bone portion. According to one aspect of the present invention, the fixation assembly includes a plurality of modular components articulatably coupled therebetween, at least one fastener interconnecting two of the plurality of components, and a clamp connector adapted for selective unilateral or multilateral bone fixation. The entire fixation assembly or components thereof can be selectively radiolucent.

In a more particular form, the present teachings provide an external fixation assembly for securing a first bone portion in fixed relationship relative to a second bone portion. The external fixation assembly includes a first radiolucent clamp assembly for connection to the first bone portion, a second radiolucent clamp assembly for connection to the second bone portion, and a radiolucent modular central body coupled to the first and second clamp assemblies. The central body includes a first intermediate connector radiolucently coupled to a second intermediate connector for relative rotational articulation about a first axis, a first end connector radiolucently coupled to the first intermediate connector for relative rotational articulation about a second axis perpendicular to the first axis, wherein the first end articulation connector is coupled to the first clamp assembly for rotation about a third axis which is orthogonal to the second axis. The central body also includes a second end connector radiolucently coupled to the second intermediate connector for relative rotational articulation about a fourth axis orthogonal to the first axis, wherein the second end connector is coupled to the second clamp assembly for rotation about a fifth axis which is orthogonal to the fourth axis. At least one of the first and second radiolucent clamp assemblies comprises a clamp connector for selective unilateral or multilateral bone fixation.

The present teachings also provide a method for externally securing a first bone portion in fixed relationship relative to a second bone portion. The method includes attaching a first clamp assembly to the first bone portion, attaching a second clamp assembly to the second bone portion, coupling at least one of the first or second clamp assemblies to a clamp connector adapted for selective unilateral or multilateral bone fixation, and connecting the first clamp assembly to the second clamp assembly with a selectively articulatable central body. The first clamp assembly, the second clamp assembly, the central body, the clamp connector or portions thereof can be selectively radiolucent.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is a perspective environmental view of an external fixation assembly according to the present teachings attached to a bone;

FIG. 2 is a plan view of an external fixation assembly according to the present teachings;

FIG. 3 is a plan view of an external fixation assembly according to the present teachings;

FIG. 4 is a plan view of an external fixation assembly according to the present teachings;

FIG. 5 is a perspective view of a central body for an external fixation assembly according to the present teachings;

FIG. 6 is a perspective view of an end connector for an external fixation assembly according to the present teachings;

FIG. 7 is a perspective view of an intermediate connector for an external fixation assembly according to the present teachings;

FIG. 8 is a front view of a central body for an external fixation assembly according to the present teachings;

FIG. 9 is a side elevational view of a central body for an external fixation assembly according to the present teachings;

FIG. 10 is plan view of an intermediate connector for an external fixation assembly according to the present teachings, shown partially cut away;

FIG. 11 is a sectional view of the intermediate connector of FIG. 10 taken along line 11-11;

FIG. 12 is a sectional view of an end connector or an external fixation assembly according to the present teachings;

FIG. 13 is a sectional view of the end connector of FIG. 12 taken along line 13-13;

FIG. 14 is plan view of a radiolucent fastener for an external fixation assembly according to the present teachings;

FIG. 15 is an end view of the radiolucent fastener of FIG. 14;

FIG. 16 is a perspective view of a radiolucent clamp assembly for an external fixation assembly according to the present teachings;

FIG. 17 is a perspective view of a radiolucent clamp assembly for an external fixation assembly according to the present teachings;

FIG. 18 is a side elevational view of a radiolucent clamp assembly for an external fixation assembly according to the present teachings;

FIG. 19 is a plan view of the clamp assembly of FIG. 18;

FIG. 20 is an elevational view of a base for the clamp assembly of FIG. 18;

FIG. 21 is cross-sectional view of the base of FIG. 20 taken along line 21-21;

FIG. 22 is a plan view of the base of FIG. 18;

FIG. 23 is a plan view of an ankle clamp assembly for the external fixation assembly according to the present teachings;

FIG. 23A is a plan view of an ankle clamp assembly for the external fixation assembly according to the present teachings;

FIG. 24 is a sectional view of the clamp assembly of FIG. 23 taken along line 24-24;

FIG. 25 is a sectional view of the clamp assembly of FIG. 23 taken along line 25-25;

FIG. 26 is a plan view of a radiolucent clamp connector for an external fixation assembly according to the present teachings;

FIG. 27 is a left end view of the clamp connector of FIG. 26;

FIG. 28 is a right end view of the clamp connector of FIG. 26;

FIG. 29 is plan view of a T-clamp assembly for an external fixation assembly according to the present teachings;

FIG. 30 is a side elevational view of the T-clamp assembly of FIG. 29;

FIG. 31 is a perspective view of a fixation construct for a fixation assembly according to the present teachings;

FIG. 32 is an environmental perspective view of a fixation assembly according to the present teachings; and

FIG. 33 is a side view of a multilateral clamp for a fixation assembly according to the present teachings.

### DETAILED DESCRIPTION

The following description is merely exemplary in nature and is in no way intended to limit the invention, its application, or uses.

Referring to FIG. 1, an exemplary external fixation assembly 100 according to the present teachings is illustrated operatively secured to a femur. Those skilled in the art will appreciate that the various teachings of the present invention are applicable to other bones. In this regard, some alternative applications are described below. The entire fixation assembly 100 or portions thereof, including entire components, can be radiolucent.

The external fixation assembly 100 is shown in an environmental view for securing a first bone portion 50 in fixed relationship relative to a second bone portion 52 during bone regeneration or other procedure of a fracture or other repair site 54. The fixation assembly 100 is illustrated to generally include a first clamp assembly 102, a second clamp assembly 104 and a preferably radiolucent central body 106. The clamp assemblies 102, 104, which can also be radiolucent or partially radiolucent, can be attached to the first and second bone portions 50, 52 respectively using bone fasteners 108. The bone fasteners 108 can be radio-opaque, such as metal screws, nails, and the like. The radiolucent central body 106 provides full unobstructed radiographic visibility of the entire repair site 54. The central body 106 can be rotationally adjusted and secured in a selected orientation relative to each of the clamp assemblies 102, 104 using set screws 113 or other fasteners that can be radio-opaque or radiolucent.

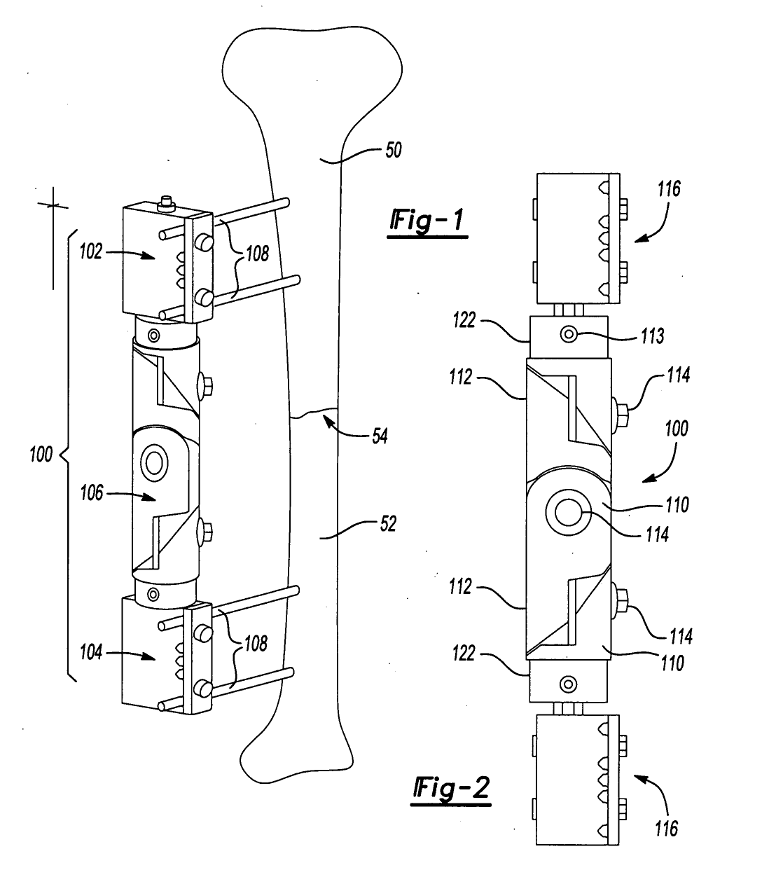

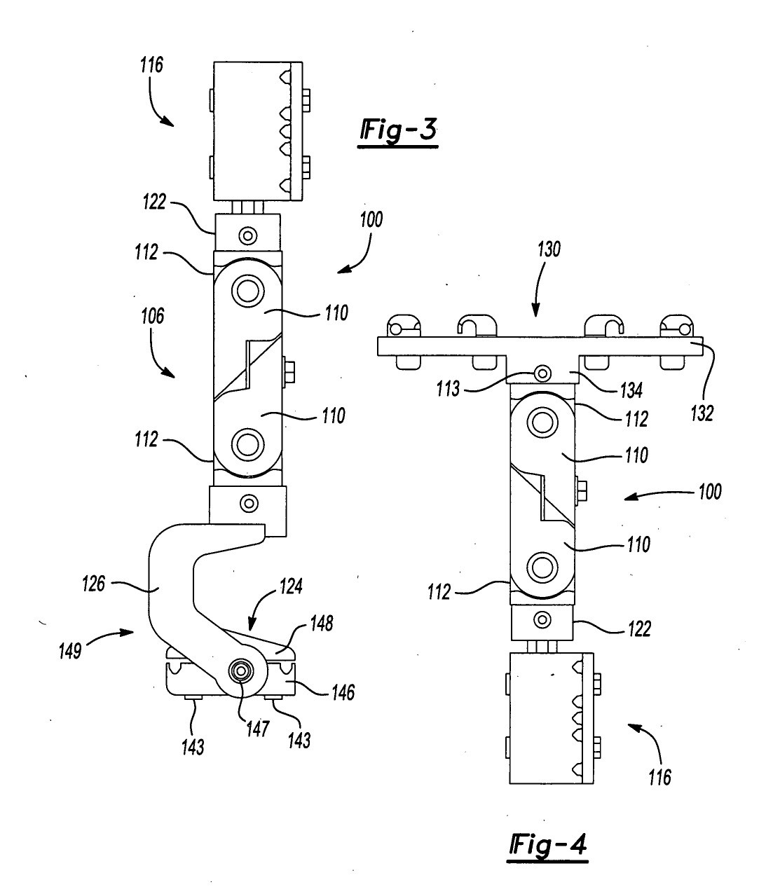

Referring to FIGS. 2-4, the external fixation assembly 100 is provided with first and second clamp assemblies 102, 104 that are adapted for various applications. The first and second clamp assemblies 102, 104 shown in FIG. 2 are straight clamp assemblies 116, and can be used, for example, for fixation of long bones with intermediate fractures, for knee fusion, or other applications. The external fixation assembly 100 illustrated in FIG. 3 includes a straight clamp assembly 116 and an ankle clamp assembly 124, and can be used, for example, for pilon fractures and other applications. The external fixation assembly 100 illustrated in FIG. 4 includes a straight clamp assembly 116 and a T-clamp assembly 130, and can be used, for example, on the proximal tibia, for ankle fusion, and other applications.

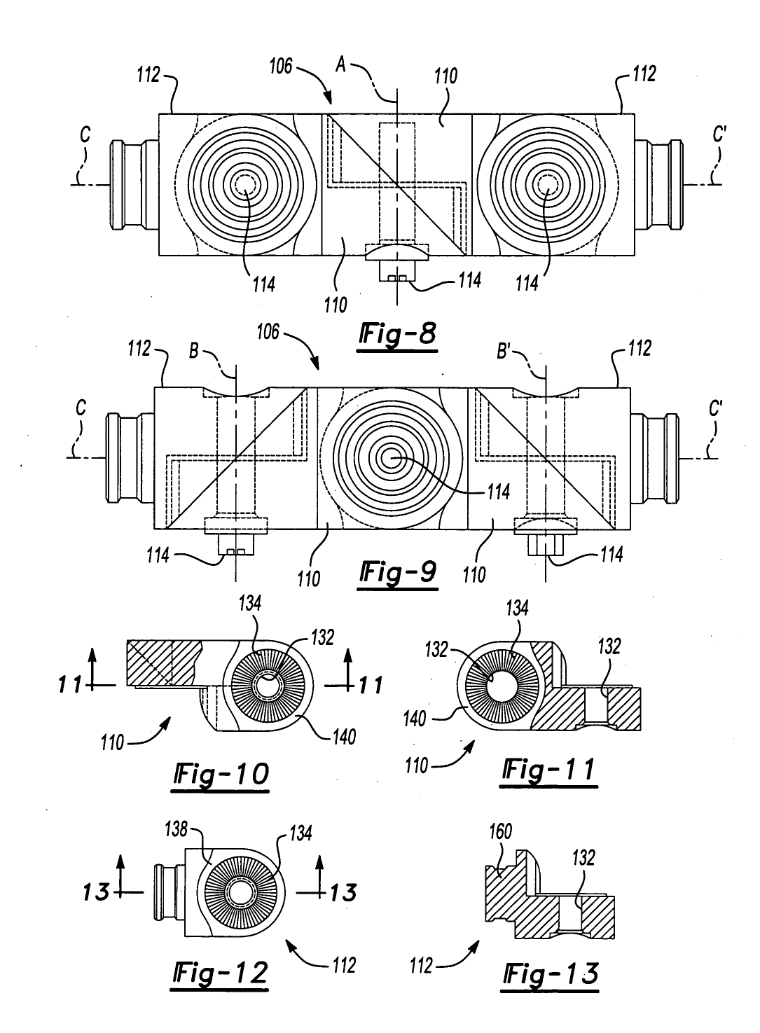

Referring to FIGS. 5, 8 and 9, the central body 106 can be modular comprising a plurality of radiolucent components that are interconnected using radiolucent fasteners 114 for selective articulation therebetween about first, second and third rotation axes A, B and B′, as will be described in detail below. The first axis A is generally orthogonal to the second and third axes B and B′. The central body 106 can also be rotated relative the first and second clamp assemblies 102, 104 about fourth and fifth rotation axes C, and C′, respectively. The fourth and fifth axes C, C′ can be collinear, as illustrated in FIGS. 5, 8 and 9, but can also be at an angle. Correspondingly, the second and third axes B, B′ can be parallel, or at an angle. The second axis B is generally orthogonal to the third axis C, and the fourth axis B′ is generally orthogonal to the fifth axis C′. Accordingly, the external fixation device 100 has multiple degrees of freedom and can be attached at any desirable orientation relative to each of the first and second bone portions 50, 52.

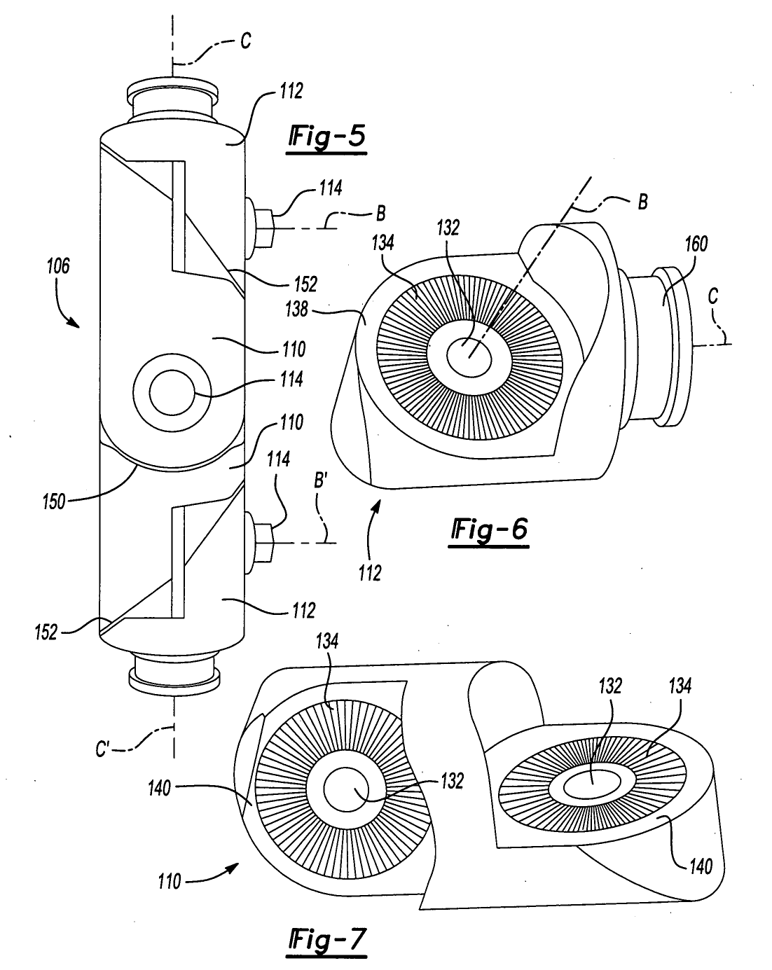

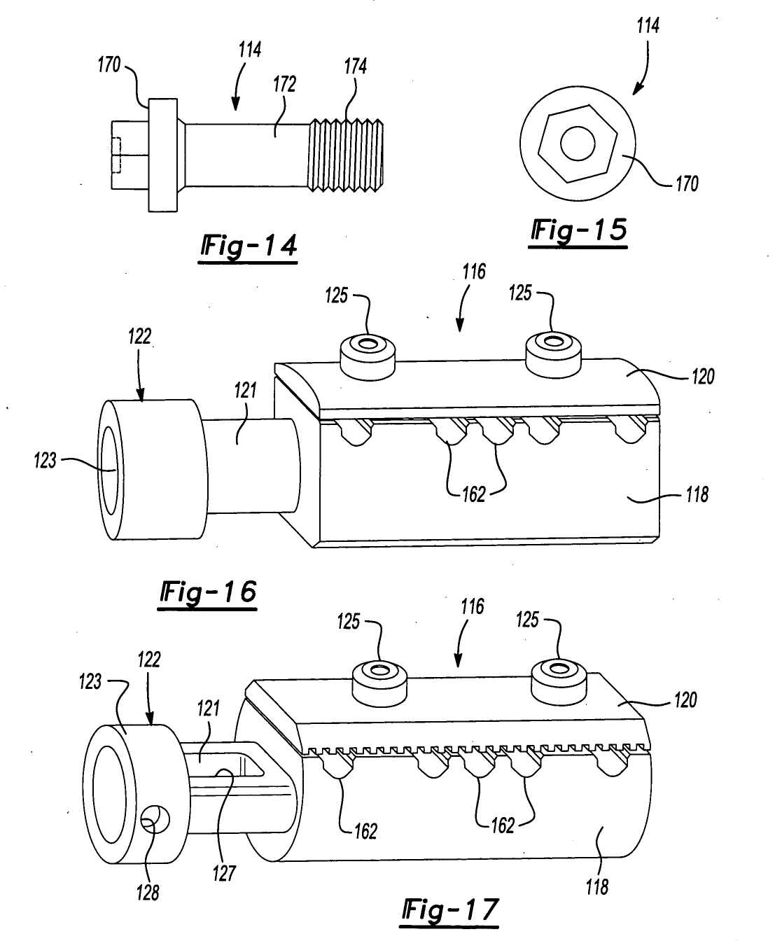

Referring to FIGS. 5, 6, 7, 14, and 15, the central body 106 comprises components that may include at least one radiolucent intermediate connector 110, and at least two radiolucent end connectors 112. The intermediate and end connectors 110, 112 can be adjustably interconnected in various combinations using radiolucent fasteners 114. Each radiolucent fastener 114 can include a head 170 adapted to engage a driver, and a body 172 that can include a threaded portion 174. The exemplary central body 106 illustrated in FIG. 5 includes a pair of adjustably interconnected intermediate connectors 110. Each intermediate connector 110 is adjustably connected to an end connector 112 by a corresponding radiolucent fastener 114. The intermediate connectors 110 define inner articulation joints 150 therebetween, and outer articulation joints 152 with the end connectors 112.

Other combinations of components will be understood to be within the scope of the present teachings. For example, the central body 106 can include one intermediate connector 110 coupled to two end connectors 112, or three intermediate connectors 110 coupled serially therebetween and to an end connector 112 at each end of the central body 106. By increasing the number of intermediate connectors 110 that are included in the central body 106, additional inner articulation joints 150 can be provided, and the length of the central body 106 can be accordingly increased.

Referring to FIGS. 5, 6, 7, and 10-13, the end connector includes an articulation surface 138 for articulation about the axis B, a fastener-receiving hole 132, and a boss 160 defining the axis of rotation C which is perpendicular to the axis B. The intermediate connector 110 includes two mutually orthogonal articulation surfaces 140, each having a fastener-receiving hole 132. One of the articulation surfaces 140 is coupled with the articulation surface 138 of the end connector 112 to define one of the outer articulation joint 152, and the other articulation surface 140 is coupled with a corresponding articulation surface 140 of another intermediate connector 110 to define the inner articulation joint 150, as shown, for example, in FIG. 5.

Each of the articulation surfaces 138, 140 can include a plurality of radial serrations 134 arranged annularly around the corresponding fastener-receiving hole 132. The serrations 134 on the opposite faces of each of the inner and outer articulation joints 150, 152 mate and interengage to provide a rotational articulation that provides a gradated index-like tactility. The serrations 134 can be formed as an integral part of the corresponding radiolucent component during the molding or other manufacturing process of making the component, and from the same radiolucent material that is used for all the components and fasteners 114 of the central body 106. The radiolucent material can be, for example, a carbon or glass fiber reinforced composite, such as the composite Orthtek® manufactured by Greene, Tweed & Co, Kulpsville, Pa. Other radiolucent materials that have suitable strength and durability characteristics may be alternatively employed. The various radiolucent components, including the components that have integral serrations 134 can be manufactured by processes developed for specific radiolucent materials by companies, such as Greene, Tweed & Co, or other companies. Such manufacturing processes may include injection molding, compression molding, thermo molding, or other suitable processes.

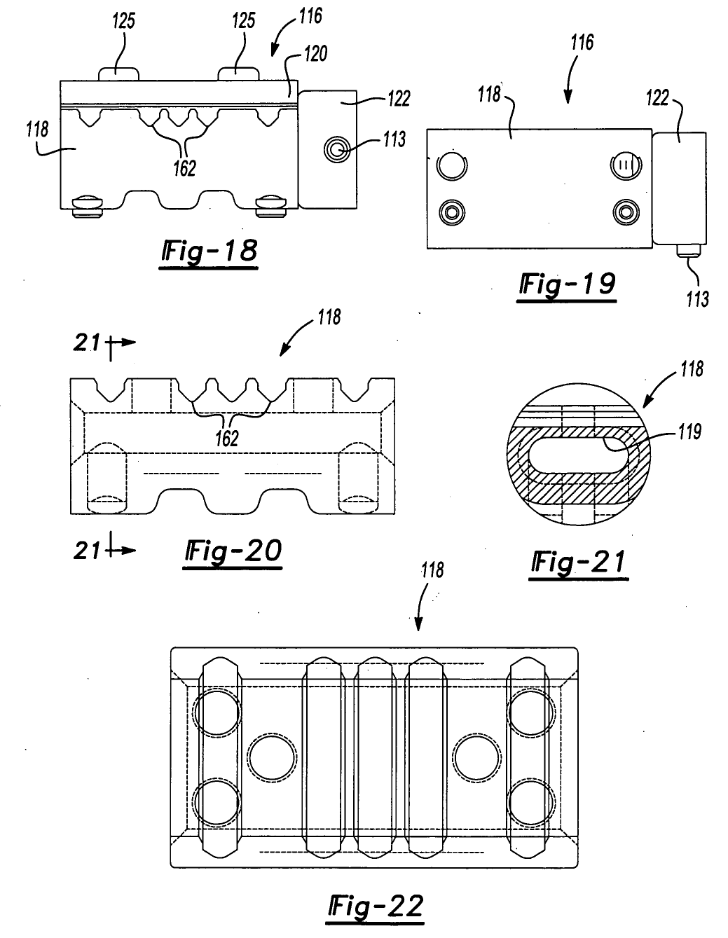

Referring to FIGS. 16, 17, 18-22, and 26-28, the straight clamp assembly 116 can include a base 118, a cover 120, and clamp fasteners 125. A clamp connector 122 can be used to connect the straight clamp assembly 116 with the central body 106. The straight clamp assembly 116 can be similar to the clamp assemblies described in co-owned U.S. Pat. Nos. 5,941,879 and 5,662,650, which are incorporated herein by reference. Depending on the application, the straight clamp assembly 116 and the clamp connector 122 can be radio-opaque, fully radiolucent, or partially radiolucent. For example, the clamp fasteners 125 and/or the clamp connector 122 can be metallic (radio-opaque), and the balance radiolucent. Alternatively, the entire straight clamp assembly 116, including the clamp fasteners 125, the base 118 and the cover 120, and the clamp connector 122, can be radiolucent for applications in which enhanced radiographic visibility is desirable in the vicinity of the straight clamp assembly 116.

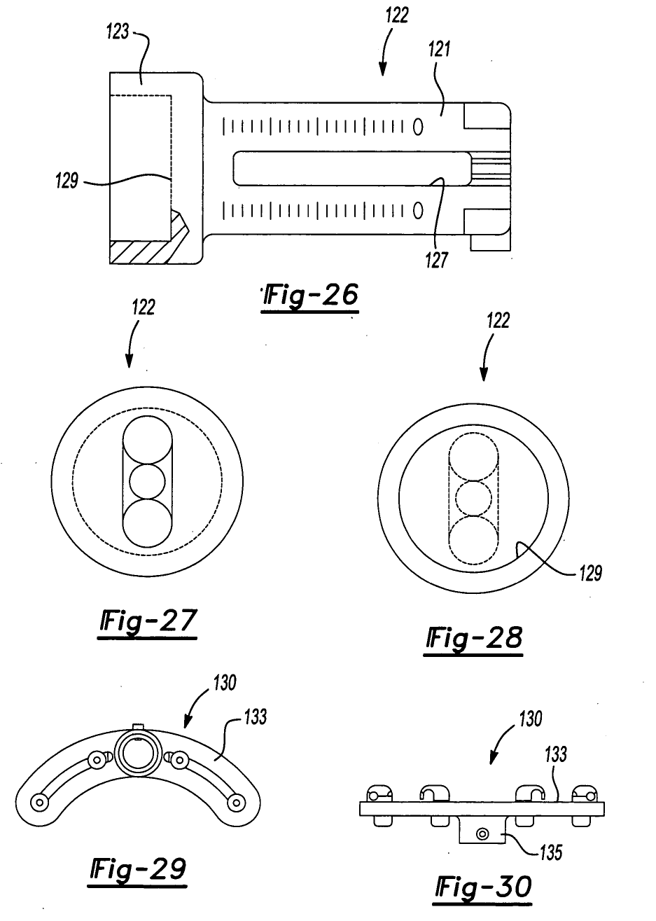

The clamp connector 122 can include a head 123 and an arm 121. The base 118 includes a channel 119 which receives the arm 121 of the clamp connector 122, thereby providing a telescopic connection that can be secured with a set screw at a desired length. Referring to the embodiment illustrated in FIG. 21, the arm 121 and the channel 119 can have mating oval or other non-circular cross-sectional shape to prevent rotation of the clamp connector 122 relative to the base 118. The arm 121 can be rail-shaped and include rails 180 defining one or more grooves 127 for engaging a set screw to provide slidable connection of limited extent, such as for intra and post-operative compression/distraction, as illustrated particularly in FIGS. 17 and 26. The head 123 of the clamp connector 122 can include a recess 129 for receiving the boss 160 of the end connector 112 and allowing relative rotation therebetween. A set screw 113 can be inserted at a hole 128 of the head 123 to secure the straight clamp assembly 116 at a desired orientation relative to the end connector 112, as illustrated in FIGS. 2, and 17-19. The straight clamp assembly 116 can include, for example, V-shaped clamping apertures 162 for receiving and compressing the bone fasteners 108 as known in the art, and illustrated in FIGS. 1 and 17.

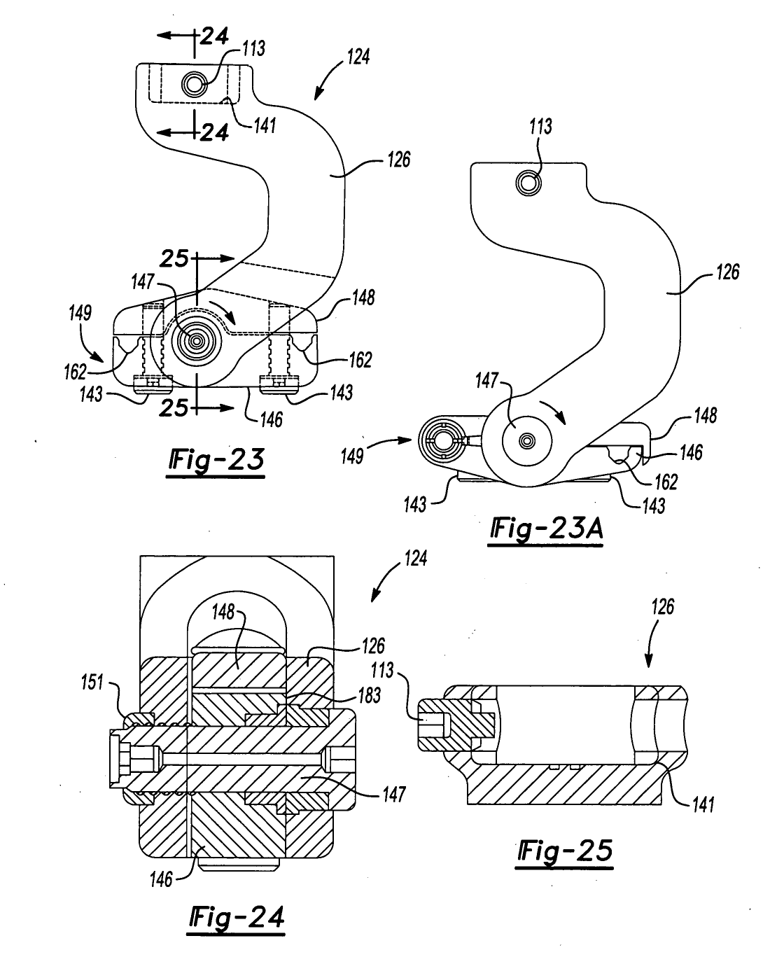

Referring to FIGS. 3 and 23-25, the ankle clamp assembly 124 can be similar to known ankle clamp assemblies, such as, for example, the one described in commonly assigned U.S. Pat. No. 6,171,308, which is incorporated herein by reference. The ankle clamp assembly 124 can include a curved ankle body 126 that articulates with an ankle clamp 149 for rotation about a pivot provided by a fastener 147, which is secured with a nut 151. The ankle clamp 149 can include an ankle clamp base 146 and an ankle clamp cover 148 that define V-shaped clamping apertures 162 therebetween, as shown in FIG. 23, for receiving and compressing the bone fasteners 108. A ball clamping aperture 163 can also be used to provide flexibility in selecting a desired orientation for the bone fasteners 108, as shown in FIG. 23A.

Similarly to the straight clamp assembly 116 discussed above, the ankle clamp assembly 124 can also be radio-opaque, fully radiolucent or partially radiolucent, as may be desired in a particular application. For example, the entire ankle clamp assembly 124, including the fastener 147, the nut 151 and clamp screws 143, can be radiolucent. Alternatively, the fastener 147 and/or the clamp screws 143 and/or the ankle clamp 149 can be radio-opaque, if desired. Additionally, serrated articulating joints 183 can be provided between the ankle body 126 and the ankle clamp 149. The serrated joints 183 can be formed on metallic or radiolucent inserts, as illustrated in FIG. 24, or can be directly molded from radiolucent material on the corresponding radiolucent components during molding of those components. The ankle body 126 can include a recess 141 for receiving the boss 160 of one of the end connectors 112 of the central body 106. A set screw 113 can be used to provide rotational connection between the ankle body 126 and the end connector 112.

Referring to FIGS. 4, 29 and 30, the T-clamp assembly 130 can be similar to T-clamp assemblies known in the art. The T-clamp assembly 130 can include a curved radiolucent body 133 with a connecting portion 135 that defines a recess for receiving the boss 160 of one of the end connectors 112 of the central body 106. A set screw 113 can be used to provide rotational connection between the body 133 of the T-clamp assembly 130 and the end connector 112. The T-clamp assembly 130 can be radio-opaque, fully radiolucent (including a radiolucent set screw 113) or partially radiolucent, as may be desired in a particular application.

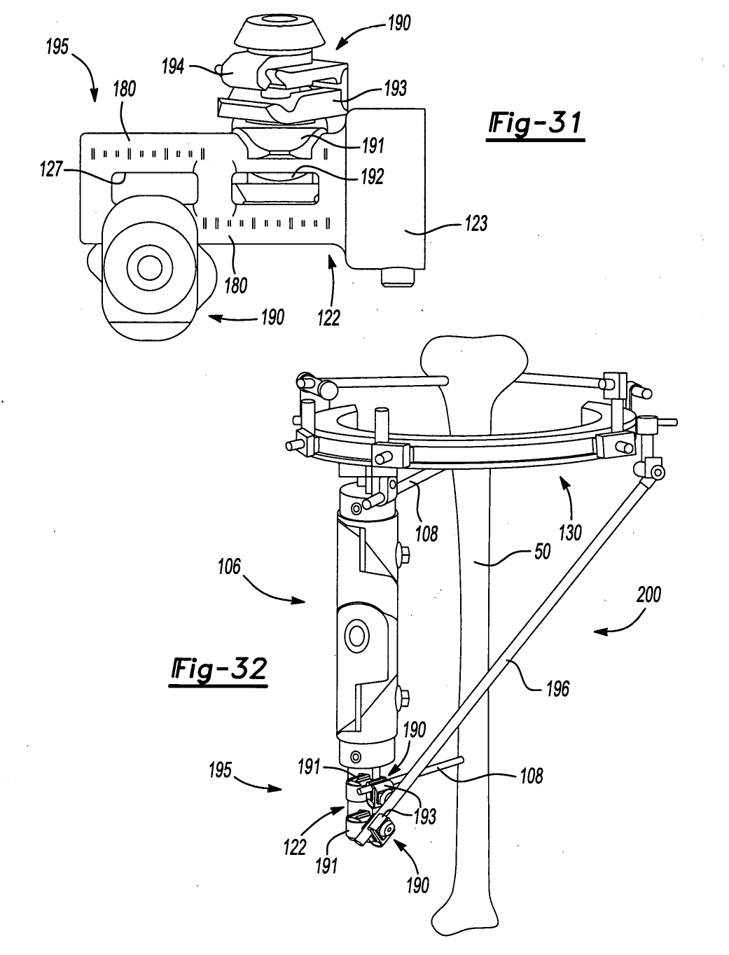

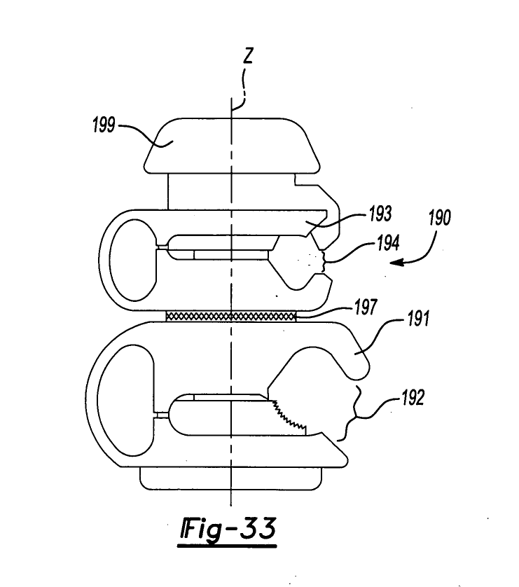

As discussed above with reference to FIG. 17, the clamp connector 122 can be use with the straight clamp assembly 116 when unilateral bone fixation is desirable. Referring to FIGS. 31-33, the clamp connector 122 can also be used independently of the straight clamp assembly 116 for hybrid fixation applications in a multidirectional fixation construct 195 that includes one or more radiolucent or metallic multilateral clamps 190, such as those available from Biomet Manufacturing Company, Warsaw, Ind., or other multi-directional clamps. For example, the clamp connector 122 can be coupled to the main body 106 at the head 123 and also coupled at the rails 180 with the multilateral clamps 190. Each multilateral clamp 190 can provide multidirectional fixation and includes a first clamp 191 having a first pair of jaws 192 that can be attached and hold one of the rails 180, and a second clamp 193 having pair of jaws 194 can be used to grasp another bar or pin that can be positioned in any orientation with respect to the rails 180. The fist and second clamps 191, 193 can articulate about an axis Z over opposite articulation surfaces 197 that can include radial serrations, and can be secured at a desired relative orientation by a fastener 199. The first and second pairs of jaws 192, 194 can define differently sized openings to accommodate pins or bars of different widths or diameters. For example, complex fractures of the tibia or other bone can be treated using the external fixation assembly 100 of FIG. 1 with the multidirectional fixation construct 195 of FIG. 31 attached thereto, for combining the rotational freedom provided by the central body 106 and the versatility provided by using the multilateral clamps 190 that allow bone fixation in any angled or rotational orientation relative to the bone, in contrast to the unilateral bone fixation provided by the straight clamp assembly 116. FIG. 32 illustrates an exemplary hybrid fixation assembly 200 that combines the central body 106, the T-clamp assembly 106, and the multidirectional fixation construct 195 that includes the clamp connector 122 and two multilateral clamps 190 coupled to the clamp connector 122 and supporting a bone screw 108 and a bar 196.

It will again be appreciated that the presents teachings are not limited to the exemplary fixation assemblies 100, 200 illustrated herein. For example, other known clamp assemblies, fastening devices, articulation connectors and clamp connectors can be used. The present teachings can be advantageously used to provide customized fixation assemblies with fully radiolucent articulation joints having radiolucent serrated surfaces and radiolucent fasteners, or other connecting devices. Appropriate components can be selected and assembled to provide a fixation assembly with a desired degree of radiographic visibility at a bone repair site and for a variety of fixation procedures.

The foregoing discussion discloses and describes merely exemplary arrangements of the present invention. One skilled in the art will readily recognize from such discussion, and from the accompanying drawings and claims, that various changes, modifications and variations can be made therein without departing from the spirit and scope of the invention as defined in the following claims.

What is claimed is:

1. A fixation assembly for securing a first bone portion in fixed relationship relative to a second bone portion, the fixation assembly comprising:
a plurality of modular components articulatably coupled therebetween;
at least one fastener interconnecting two of the plurality of components; and
a clamp connector adapted for selective unilateral or multilateral bone fixation.

2. The fixation assembly of claim 1, wherein selectively the entire fixation assembly or portions thereof are radiolucent.

3. The fixation assembly of claim 2, wherein the plurality of modular components comprises:
a first clamp assembly for connection to the first bone portion;
a second clamp assembly for connection to the second bone portion; and
a central body for coupling the first clamp assembly in a fixed orientation relative to the second clamp assembly.

4. The fixation assembly of claim 3, wherein the central body comprises a plurality of central body components selectively mutually articulatable for fixating the first and second bone portions in a selected orientation.

5. The fixation assembly of claim 4, wherein the central body components are coupled by radiolucent fasteners.

6. The external fixation assembly of claim 5, wherein each central body component comprises at least one articulation surface having a plurality of radiolucent radial serrations around a fastener-receiving aperture.

7. The external fixation assembly of claim 6, wherein the serrations define an indexable rotation between articulating central body components.

8. The fixation assembly of claim 7, wherein the plurality of central body components comprises at least one intermediate connector and at least two end connectors.

9. The fixation assembly of claim 8, further comprising a telescopic arm coupled to the central body and to one of the first and second clamp assemblies.

10. The external assembly of claim 8, wherein one of the first and second clamp assemblies comprises an ankle clamp assembly.

11. The fixation assembly of claim 8, wherein one of the first and second clamp assemblies comprises a straight clamp assembly.

12. The fixation assembly of claim 11, wherein the straight clamp assembly includes a base portion and a cover, the base and the cover cooperatively defining clamping apertures for engaging bone fasteners.

13. The fixation assembly of claim 12, wherein at least one clamping aperture comprises a ball clamping aperture.

14. The fixation assembly of claim 12, wherein at least one clamping aperture comprises a V-shaped clamping aperture.

15. The fixation assembly of claim 11, wherein the straight clamp assembly is modularly coupled to the central body with the clamp connector.

16. The fixation assembly of claim 15, wherein the clamp connector is telescopically adjustable relative to the straight clamp assembly.

17. The fixation assembly of claim 8, wherein the intermediate connector provides articulation about two orthogonal rotation axes.

18. The fixation assembly of claim 2, wherein the radiolucent portions comprise carbon.

19. The fixation assembly of claim 1, wherein the clamp connector comprises an arm for unilateral bone fixation.

20. The fixation assembly of claim 19, further comprising a straight clamp assembly coupled to the arm of the clamp connector.

21. The fixation assembly of claim 19, wherein the arm comprises at least one rail for multilateral bone fixation.

22. The fixation assembly of claim 21, further comprising a multilateral clamp coupled to the at least one rail of the clamp connector.

23. An external fixation assembly for securing a first bone portion in fixed relationship relative to a second bone portion, the external fixation assembly comprising:
a first radiolucent clamp assembly for connection to the first bone portion;
a second radiolucent clamp assembly for connection to the second bone portion; and
a radiolucent modular central body coupled to the first and second clamp assemblies, wherein the central body comprises:
a first intermediate connector radiolucently coupled to a second intermediate connector for relative rotational articulation about a first axis;
a first end connector radiolucently coupled to the first intermediate connector for relative rotational articulation about a second axis perpendicular to the first axis, the first end connector coupled to the first clamp assembly for rotation about a third axis orthogonal to the second axis; and
a second end connector radiolucently coupled to the second intermediate connector for relative rotational articulation about a fourth axis orthogonal to the first axis, the second end connector coupled to the second clamp assembly for rotation about a fifth axis orthogonal to the fourth axis,
wherein at least one of the first and second radiolucent clamp assemblies comprises a clamp connector for selective unilateral or multilateral bone fixation.

24. The external fixation assembly of claim 23, wherein each of the first and second intermediate and end connectors comprises at least one articulation surface having a plurality of radial serrations around a fastener-receiving aperture.

25. The external fixation assembly of claim 24, wherein the plurality of radial serrations define an indexable rotation between mutually articulatable components.

26. The external fixation assembly of claim 23, further comprising at least one multilateral clamp coupled to a rail of the clamp connector.

27. The external fixation assembly of claim 23, wherein one of the first or second clamp assemblies comprises a straight clamp assembly coupled to an arm of the clamp connector.

28. A method for externally securing a first bone portion in fixed relationship relative to a second bone portion, the method comprising:
attaching a first clamp assembly to the first bone portion;
attaching a second clamp assembly to the second bone portion;
coupling at least one of the first or second clamp assemblies to a clamp connector adapted for selective unilateral or multilateral bone fixation; and
connecting the first clamp assembly to the second clamp assembly with a selectively articulatable central body.

29. The method of claim 28, wherein the first clamp assembly, the second clamp assembly, the central body, the clamp connector or portions thereof are selectively radiolucent.

30. The method of claim 29, wherein the central body comprises a plurality of modular components radiolucently fastened therebetween.

31. The method of claim 29, wherein each of the modular components includes at least one radiolucent serrated articulation surface.
